(19) United States
(12) Patent Application Publication
(10) Pub. No.: US 2024/0307401 A1
(43) Pub. Date: Sep. 19, 2024

(54) TREATMENT OF JAK-INHIBITION-RESPONSIVE DISORDERS WITH PRODRUGS OF JAK INHIBITORS

(71) Applicant: SUN PHARMACEEUTICAL INDUSTRIES, INC., Princeton, NJ (US)

(72) Inventor: Adam J. Morgan, Ashland, MA (US)

(21) Appl. No.: 18/682,339

(22) PCT Filed: Aug. 12, 2022
(86) PCT No.: PCT/US22/40190

§ 371 Date: Feb. 8, 2024

Related U.S. Application Data
(60) Provisional application No. 63/232,538, filed on Aug. 12, 2021.
(60) Provisional application No. 63/233,059, filed on Aug. 13, 2021.

(51) Int. Cl. A61K 31/519; A61K 31/506

(52) U.S. Cl. CPC A61K 31/519; A61K 31/506

(57) ABSTRACT

A method of treating a JAK-inhibition-responsive condition in a subject in need thereof, the method comprising: administering to the subject an effective amount of a compound represented by structural formulas (I) or (II), as described herein, or a pharmaceutically acceptable salt thereof.

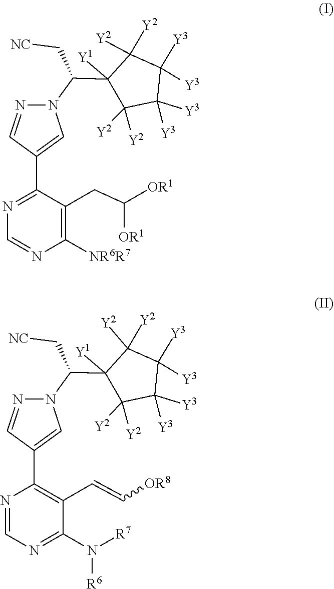

### RELATED APPLICATIONS

This application claims the benefit of U.S. Provisional Application No. 63/232,538, filed on Aug. 12, 2021 and U.S. Provisional Application No. 63/233,059, filed on Aug. 13, 2021. The entire teachings of the above applications are incorporated herein by reference.

### BACKGROUND OF THE INVENTION

Ruxolitinib phosphate is a heteroaryl-substituted pyrrolo[2,3-d]pyrimidine, also known as 3(R)-cyclopentyl-3-[4-(7H-pyrrolo[2,3-d]pyrimidin-4-yl)-1H-pyrazol-1-yl]propanenitrile phosphate, and as (R)-3-(4-(7H-pyrrolo[2,3-d]pyrimidin-4-yl)-1H-pyrazol-1-yl)-3-cyclopentylpropanenitrile phosphate, which inhibits Janus Associated Kinases (JAKs) JAK1 and JAK2. These kinases mediate the signaling of a number of cytokines and growth factors important for hematopoiesis and immune function. JAK signaling involves recruitment of STATs (signal transducers and activators of transcription) to cytokine receptors, activation and subsequent localization of STATs to the nucleus leading to modulation of gene expression.

Ruxolitinib phosphate has been approved in the US and Europe for the treatment of myelofibrosis and for the treatment of polycythemia vera. Ruxolitinib is currently in clinical trials for the treatment of graft-versus-host disease and other conditions.

A deuterated analog of ruxolitinib, (R)-3-(4-(7H-pyrrolo[2,3-d]pyrimidin-4-yl)-1H-pyrazol-1-yl)-3-(cyclopentyl-2,2,3,3,4,4,5,5-d₈)propanenitrile (referred to herein as CTP-543), is a potent selective inhibitor of Janus kinases JAK1 and JAK2. The compound is disclosed in International Patent Applications WO 2013/188783A1, WO 2017/192905A1, and WO 2020/163653A1. CTP-543 is currently being investigated in human clinical trials and has at certain doses been shown to stimulate hair growth in patients suffering from alopecia areata. Despite the beneficial activities of ruxolitinib and CTP-543, there is a continuing need for new compounds that inhibit JAK1 and JAK2 and that can be used to treat the aforementioned diseases and conditions.

### SUMMARY OF INVENTION

In an example embodiment, the present invention is a method of treating a JAK-inhibition-responsive condition in a subject in need thereof. The method comprises: administering to the subject an effective amount of a compound represented by structural Formula (I) or (II):

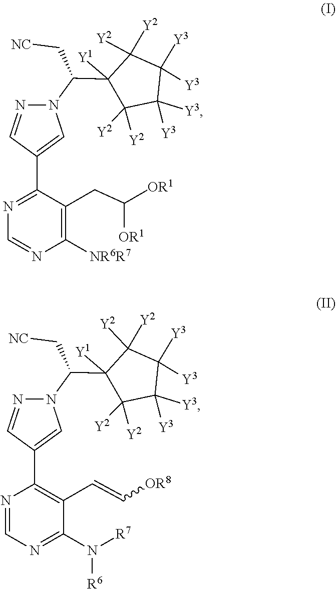

or a pharmaceutically acceptable salt thereof, wherein for each occurrence independently:
Y¹ is hydrogen or deuterium;
Y² is the same and is hydrogen or deuterium;
Y³ is the same and is hydrogen or deuterium;
R⁸ is a C₁-C₆ alkyl;
each R¹ independently is a C₁-C₆ alkyl, or the two R¹s, taken together with the oxygen atoms to which they are attached, form a 5- or 6-membered heterocyclic ring; and R⁶ and R⁷, for each occurrence are independently selected from H and a nitrogen protecting group.

In another example embodiment, the present invention is a method of treating a JAK-inhibition-responsive condition in a subject in need thereof. The method comprises: administering to the subject an effective amount of a pharmaceutical composition comprising a pharmaceutically acceptable carrier or diluent and a compound represented by structural Formula (I) or (II):

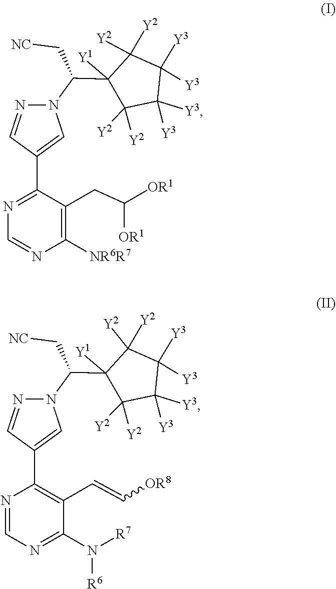

or a pharmaceutically acceptable salt thereof, wherein, for each occurrence independently: Y¹ is hydrogen or deuterium; Y² is the same and is hydrogen or deuterium; Y³ is the same and is hydrogen or deuterium; R⁸ is a C₁-C₆ alkyl; each R¹ independently is a C₁-C₆ alkyl, or the two R¹s, taken together with the oxygen atoms to which they are attached, form a 5- or 6-membered heterocyclic ring; and R⁶ and R⁷, for each occurrence are independently selected from H and a nitrogen protecting group.

### DETAILED DESCRIPTION OF THE INVENTION

As described herein, certain prodrug compounds of ruxolitinib and deuterated analogs of ruxolitinib represented by either Formula (I) or Formula (II):

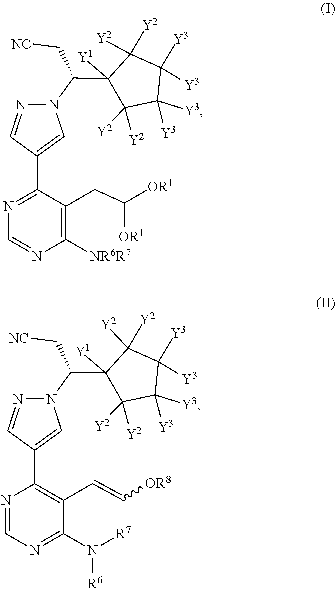

or a pharmaceutically acceptable salt thereof, upon oral administration to a mammalian subject, can be metabolized in the subject's body (e.g., in the stomach, under the acidic conditions) into active metabolites represented by Formula (III), below, such as ruxolitinib and its deuterated analogs (e.g., CTP-543). The values and example values of the variables in Formulas (I) and (II) are defined below.

Thus, a compound of Formula (I) can undergo the following transformation, when orally administered to a mammalian subject:

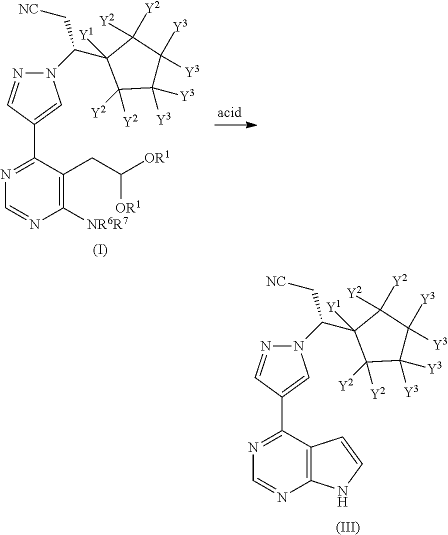

The compound of Formula (II) can undergo the following transformation when orally administered to a mammalian subject:

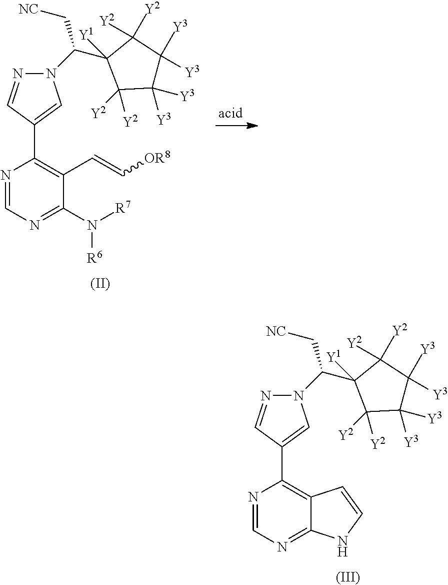

In one example embodiment, prodrug compounds of ruxolitinib and deuterated analogs of ruxolitinib are represented by Formula (IV):

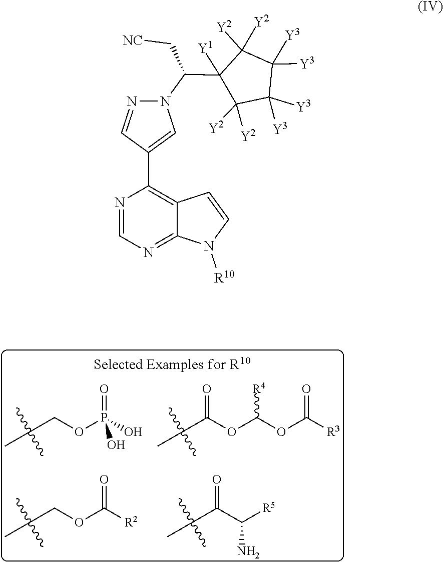

In Formula (IV), R¹⁰ can be any nitrogen-protecting group, and in certain embodiments is a group that can be cleaved in vivo.

In Formula (IV), the values of Y¹, Y², and Y³ are as described above with respect to Formulas (I) and (II); R² and R³, each independently, is a C₁-C₆ alkyl, a C₆-C₁₈ aryl, a (C₆-C₁₈)aryl-(C₁-C₃)alkyl, a 5-18-member heteroaryl, a C₃-C₆ cycloalkyl, a 5-7-member heterocyclyl; an —O—(C₁-C₆)alkyl; an —N-(mono or di)(C₁-C₆)alkyl; or a —O—(C₆-C₁₈)aryl, each of which can be optionally substituted; and R⁴ and R⁵, each independently, is a C₁-C₆ alkyl, a C₆-C₁₈ aryl, a (C₆-C₁₈)aryl-(C₁-C₃)alkyl, a 5-18-member heteroaryl, a C₃-C₆ cycloalkyl, or a 5-7-member heterocyclyl, each of which can be optionally substituted.

An example embodiment of compounds of Formula (IV) is a compound represented by structural formula (IV A):

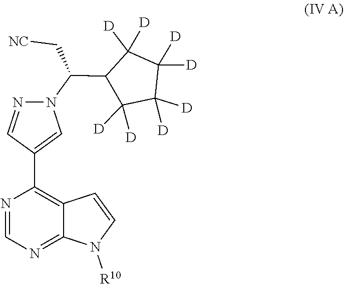

R¹⁰ can be any nitrogen-protecting group, and in certain embodiments is a group that can be cleaved in vivo. Permitted values of R¹⁰ include but are not limited to those listed above with respect to Formula (IV) as well as protecting groups selected from pivaloyloxymethyl (POM), 2-(trimethylsilyl)ethoxymethyl (SEM), benzyl (Bn), p-methoxybenzyl (PMB), 3,4-dimethoxybenzyl (DMPM), 2,4-dimethoxybenzyl, benzenesulfonyl, tosyl (Ts), t-butoxycarbonyl (BOC), methoxycarbonyl (MOC), benzyloxycarbonyl (CBz), 1-naphthalenesulfonate (1-napsyl), 4-nitrobenzenesulfonyl (p-nosyl), and 2,4,6-trimethylphenylsulfonyl.

### Definitions

As used herein, the term “prodrug” refers to a compound that metabolizes, under physiological conditions in vivo (e.g., acid conditions, such as physiological conditions having a pH of less than 4), into an active pharmaceutical ingredient, also referred herein as an “active metabolite.” For example, when compounds of Formula (I) and Formula (II) are administered orally, they can be metabolized in the mammalian body (e.g., in the stomach) into an active metabolite represented by Formula (III). Example of active metabolites represented by Formula (III) include ruxolitinib and CTP-543.

The term “treat” means decrease, suppress, attenuate, diminish, arrest, or stabilize the development or progression of a disease (e.g., a disease or disorder delineated herein), lessen the severity of the disease or improve the symptoms associated with the disease. For example, treatment of a hair loss disorder includes regrowth of hair, prevention of further hair loss, or diminishing the rate of hair loss.

“Hair loss disorder” means any condition or disorder that results in loss of hair on one or more areas of the body. Hair loss disorders include, without limitation, androgenetic alopecia, alopecia areata, telogen effluvium, alopecia areata, alopecia totalis, and alopecia universalis.

The term “mammal”, as used herein, includes humans, as well as non-human mammals such as cats, dogs, sheep, cattle, pigs, goats, non-human primates (including monkeys and apes) and the like.

As used herein, an “effective amount” of a prodrug defined by structural Formulas (I) and (II) described herein is an amount sufficient to produce a therapeutically effective amount of its corresponding active metabolite upon oral administration.

As used herein, a “therapeutically effective amount” is an amount sufficient to treat the target condition or disorder.

The term “alkyl” refers to a monovalent saturated hydrocarbon group. C₁-C₆ alkyl is an alkyl having from 1 to 6 carbon atoms. In some embodiments, an alkyl may be linear or branched. In some embodiments, an alkyl may be primary, secondary, or tertiary. Non-limiting examples of alkyl groups include methyl; ethyl; propyl, including n-propyl and isopropyl; butyl, including n-butyl, isobutyl, sec-butyl, and 1-butyl; pentyl, including, for example, n-pentyl, isopentyl, and neopentyl; and hexyl, including, for example, n-hexyl and 2-methylpentyl. Non-limiting examples of primary alkyl groups include methyl, ethyl, n-propyl, n-butyl, n-pentyl, and n-hexyl. Non-limiting examples of secondary alkyl groups include isopropyl, sec-butyl, and 2-methylpentyl. Non-limiting examples of tertiary alkyl groups include 1-butyl.

Nitrogen protecting groups, also referred to as amine protecting groups, are well known in the art and include those described in detail in Protecting Groups in Organic Synthesis, T. W. Greene and P. G. M. Wuts, 3^{rd} edition, John Wiley & Sons, 1999, incorporated herein by reference. For example, nitrogen protecting groups such as amide groups include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamina)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfa)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), -trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fern), N-2-picolylamino N′-oxide, N-1,1-dimethylthiomethyleneamine, N-benzylideneamine, N-p-methoxybenzylideneamine, N-diphenylmethyleneamine, N-[(2-pyridl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In particular embodiment, nitrogen-protecting groups are acid labile. For example, N-protecting groups are those that would deprotect in the stomach. Examples of acid-labile group are groups that are >80% deprotected in 30 minutes at pH 2.0 in aqueous media. Such groups include 1-butoxycarbonyl (Boc), triflyl (Tf, SO₂—CF₃), trifluoroacetyl (F₃—Ac), and trityl (Tr, CPh₃). In one example embodiment, the nitrogen-protecting group is t-Boc.

The term “heterocyclic group” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

It will be recognized that some variation of natural isotopic abundance occurs in a synthesized compound depending upon the origin of chemical materials used in the synthesis. Thus, a preparation of ruxolitinib will inherently contain small amounts of deuterated isotopologues. The concentration of naturally abundant stable hydrogen and carbon isotopes, notwithstanding this variation, is small and immaterial as compared to the degree of stable isotopic substitution of the deuterated compounds of this invention. See, for instance, Wada, E et al., Seikagaku, 1994, 66:15; Gannes, L Z et al., Comp Biochem Physiol Mol Integr Physiol, 1998, 119:725.

In any of the compounds described herein, any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. However, in certain embodiments where stated, when a position is designated specifically as “H” or “hydrogen”, the position has at least 80%, at least 90%, at least 95%, at least 96%, at least 97%, at least 98%, or at least 99% hydrogen. In some embodiments, where specifically stated, when a position is designated specifically as “H” or “hydrogen”, the position incorporates ≤20% deuterium, ≤10% deuterium, ≤5% deuterium, ≤4% deuterium, ≤3% deuterium, ≤2% deuterium, or ≤1% deuterium. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium). The amount of deuterium incorporation at a designated position may be measured by analytical methods known to one of ordinary skill in the art, for example, by proton NMR.

The term “isotopic enrichment factor” as used herein means the ratio between the isotopic abundance and the natural abundance of a specified isotope.

In other embodiments, a deuterated compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In some embodiments, in a compound of this invention, each designated deuterium position (or atom) has deuterium incorporation of at least 52.5%. In some embodiments, in a compound of this invention, each designated deuterium position has deuterium incorporation of at least 60%. In some embodiments, in a compound of this invention, each designated deuterium position has deuterium incorporation of at least 67.5%. In some embodiments, in a compound of this invention, each designated deuterium position has deuterium incorporation of at least 75%. In some embodiments, in a compound of this invention, each designated deuterium position has deuterium incorporation of at least 80%. In some embodiments, in a compound of this invention, each designated deuterium position has deuterium incorporation of at least 85%. In some embodiments, in a compound of this invention, each designated deuterium position has deuterium incorporation of at least 90%. In some embodiments, in a compound of this invention, each designated deuterium position has deuterium incorporation of at least 95%. In some embodiments, in a compound of this invention, each designated deuterium position has deuterium incorporation of at least 97%. In some embodiments, in a compound of this invention, each designated deuterium position has deuterium incorporation of at least 98%. In some embodiments, in a compound of this invention, each designated deuterium position has deuterium incorporation of at least 99%. In some embodiments, in a compound of this invention, each designated deuterium position has deuterium incorporation of at least 99.5%.

The term “isotopologue” refers to a species in which the chemical structure differs from any of the compounds described herein only in the isotopic composition thereof.

The term “compound,” when referring to a deuterated compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. In certain embodiments, the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of any of the compounds described herein. A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt, such as a phosphate salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In a particular embodiment, pharmaceutically acceptable salts of the compounds represented by structural formulas (I) and (II) are addition salts formed with the following acids:

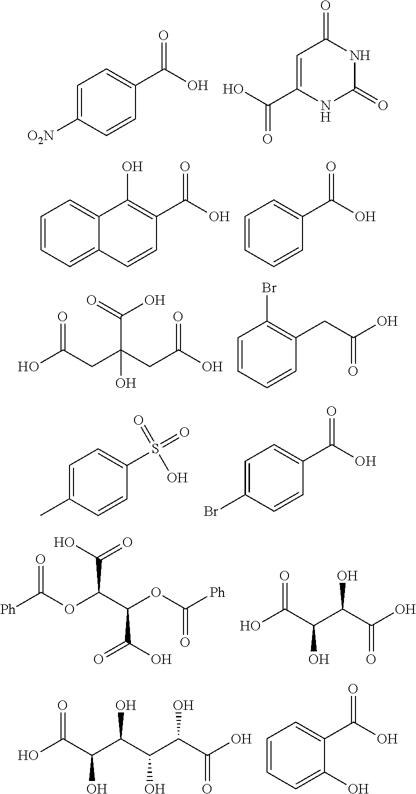

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “Stereoisomer” refers to both enantiomers and diastereomers. “Tert” and “t-” each refer to tertiary. “US” refers to the United States of America.

“Substituted with deuterium” refers to the replacement of one or more hydrogen atoms with a corresponding number of deuterium atoms.

### Compounds

In one aspect, the invention provides compounds and pharmaceutically-acceptable salts thereof.

In one embodiment, the invention provides compounds of Formula (I):

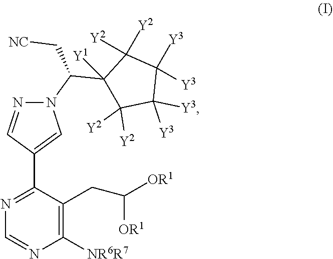

or a pharmaceutically acceptable salt thereof,
   wherein, for each occurrence independently:
Y¹ is hydrogen or deuterium; each Y² is the same and is hydrogen or deuterium; each Y³ is the same and is hydrogen or deuterium; each R¹ independently is a C₁-C₆ alkyl, or the two R¹s, taken together with the oxygen atoms to which they are attached, form a 5- or 6-membered heterocyclic ring; and R⁶ and R⁷, for each occurrence independently, are independently selected from H and a nitrogen protecting group. In certain embodiments, the nitrogen protecting group is an acid-labile protecting group. In certain embodiments, the nitrogen protecting group is not t-butoxycarbonyl (Boc), triflyl (Tf, SO₂—CF₃), trifluoroacetyl (F₃—Ac), or trityl (Tr, CPh₃). In certain embodiments, at least one of Y² and Y³ is deuterium. In certain embodiments, both Y² and Y³ are deuterium. In certain embodiments, Y¹ is hydrogen and both Y² and Y³ are deuterium.

In certain embodiments, each R¹ is the same. In certain embodiments, at least one R¹ is not methyl. In certain embodiments, at least one R¹ is not ethyl.

In another embodiment, the invention provides compounds of Formula (II):

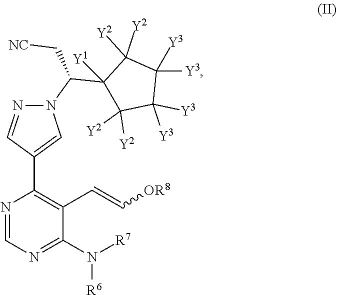

or a pharmaceutically acceptable salt thereof,
wherein, for each occurrence independently:
Y¹ is hydrogen or deuterium; Y² is the same and is hydrogen or deuterium; Y³ is the same and is hydrogen or deuterium; R⁸ is a C₁-C₆ alkyl; and R⁶ and R⁷, for each occurrence independently, are independently selected from H and a nitrogen protecting group.

In certain embodiments, R⁸ is not methyl. In certain embodiments, R⁸ is not ethyl.

In another embodiment, the invention provides compounds of Formula (IV):

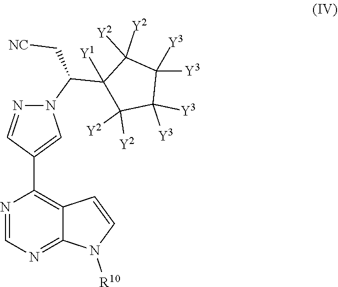

in which the values of Y¹, Y², and Y³ are as described above with respect to Formulas (I) and (II); and
R¹⁰ is any nitrogen-protecting group. In certain embodiments, R¹⁰ is a group that can be cleaved in vivo. In certain embodiments, R¹⁰ is selected from the following groups:

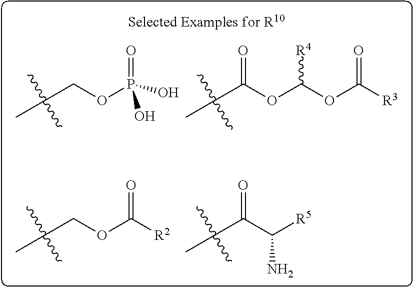

in which R² and R³, each independently, is a C₁-C₆ alkyl, a C₆-C₁₈ aryl, a (C₆-C₁₈)aryl-(C₁-C₃)alkyl, a 5-18-member heteroaryl, a C₃-C₆ cycloalkyl, a 5-7-member heterocyclyl; an —O—(C₁-C₆)alkyl; an —N-(mono or di)(C₁-C₆)alkyl; or a —O—(C₆-C₁₈)aryl, each of which can be optionally substituted; and R⁴ and R⁵, each independently, is a C₁-C₆ alkyl, a C₆-C₁₈ aryl, a (C₆-C₁₈)aryl-(C₁-C₃)alkyl, a 5-18-member heteroaryl, a C₃-C₆ cycloalkyl, or a 5-7-member heterocyclyl, each of which can be optionally substituted.

### Pharmaceutical Compositions

In another aspect, the invention provides a pharmaceutical composition of a compound of any of Formulae (I), (II), or (IV).

In one embodiment, the invention provides a pharmaceutical composition of a compound of Formula (I), or a pharmaceutically acceptable salt thereof, the pharmaceutical composition comprising a compound of Formula (I), or a pharmaceutically acceptable salt thereof, and a pharmaceutically acceptable carrier.

In one embodiment, the invention provides a pharmaceutical composition of a compound of Formula (II), or a pharmaceutically acceptable salt thereof, the pharmaceutical composition comprising a compound of Formula (II), or a pharmaceutically acceptable salt thereof, and a pharmaceutically acceptable carrier. In one embodiment, the invention provides a pharmaceutical composition of a compound of Formula (IV), or a pharmaceutically acceptable salt thereof, the pharmaceutical composition comprising a compound of Formula (IV), or a pharmaceutically acceptable salt thereof, and a pharmaceutically acceptable carrier.

### Methods of Treatment

In one aspect, the invention provides a method for treating a “JAK-inhibition-responsive condition” (e.g., a disease or disorder that can be treated by compounds that inhibit the activity of a JAK (JAK1 and/or JAK2)), the method comprising administering to a mammalian subject an effective amount of a compound of Formula (I) or a compound of Formula (II):

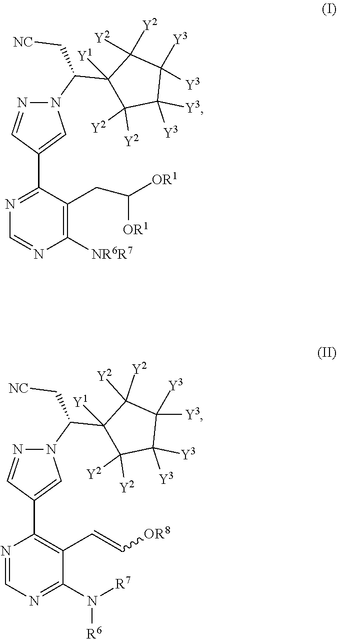

or a pharmaceutically acceptable salt thereof, wherein, for each occurrence independently:
Y¹ is hydrogen or deuterium; Y² is the same and is hydrogen or deuterium; Y³ is the same and is hydrogen or deuterium; R⁸ is a C₁-C₆ alkyl; each R¹ independently is a C₁-C₆ alkyl, or the two R¹s, taken together with the oxygen atoms to which they are attached, form a 5- or 6-membered heterocyclic ring; and R⁶ and R⁷, for each occurrence independently, are independently selected from H and a nitrogen protecting group.

In certain embodiments, the method of treatment comprises administering an effective amount of a compound of Formula (I) or Formula (II), or a pharmaceutically acceptable salt thereof defined above to a subject in need thereof.

In certain embodiments of Formula (I) or Formula (II), Y¹ is hydrogen and each of Y² and Y³ is deuterium. In certain embodiments of Formula (I) or Formula (II), Y¹ is deuterium and each of Y² and Y³ is deuterium. In certain embodiments of Formula (I) or (II), each of Y¹, Y², and Y³ is hydrogen. In certain embodiments of Formula (I), each R¹ is methyl or ethyl. In certain embodiments of Formula (I), both R¹s are methyl. In certain embodiments of Formula (I), both R¹s are ethyl. In certain embodiments of Formula (I), each R⁶ is H. In certain embodiments of Formula (I), one R⁶ is H and one R⁶ is a nitrogen protecting group (NPG). In certain embodiments of Formula (I), each R⁶ is a nitrogen protecting group. In certain embodiments of Formula (II), R⁸ is methyl or ethyl. In certain embodiments of Formula (II), both R⁶ and R⁷ are H. In certain embodiments of Formula (II), one of R⁶ and R⁷ is H and the other is a nitrogen protecting group.

In certain embodiments, the protecting group of the compounds described herein is selected from t-butoxycarbonyl (Boc), triflyl (Tf, SO₂—CF₃), trifluoroacetyl (F₃—Ac), and trityl (Tr, CPh₃). In certain embodiments, the protecting group is a t-butoxycarbonyl group. In certain embodiments, the deuterium incorporation at each position designated as deuterium is at least 90%, at least 95%, or at least 97%.

In certain embodiments, the compound of Formula I is Compound (I-1d):

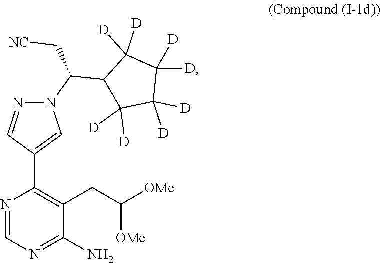

or a pharmaceutically acceptable salt thereof. As described herein, another embodiment of the invention is a composition comprising a Compound (I-1d), or a pharmaceutically acceptable salt thereof and optionally a pharmaceutically acceptable carrier. In certain embodiments, the deuterium incorporation at each position designated as deuterium in Compound (I-1d) is at least 90%, at least 95%, or at least 97%.

In certain embodiments, the compound of Formula I is Compound (I-2d):

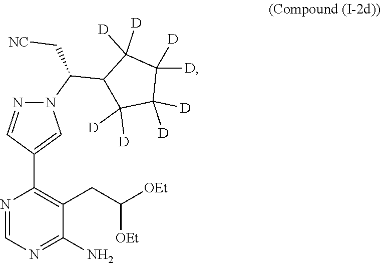

or a pharmaceutically acceptable salt thereof. As described herein, another embodiment of the invention is a composition comprising a Compound (I-2d), or a pharmaceutically acceptable salt thereof an optionally a pharmaceutically acceptable carrier. In certain embodiments, the deuterium incorporation at each position designated as deuterium in Compound (I-2d) is at least 90%, at least 95%, or at least 97%.

In certain embodiments, the compound of Formula I is Compound (I-1):

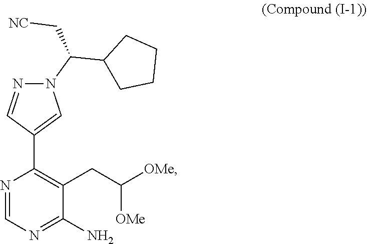 [SMILES: COC(Cc1c(N)ncnc1-c1cnn([C@H](CC#N)C2CCCC2)c1)OC]

or a pharmaceutically acceptable salt thereof. As described herein, another embodiment of the invention is a composition comprising a Compound (I-1), or a pharmaceutically acceptable salt thereof an optionally a pharmaceutically acceptable carrier. In certain embodiments, the deuterium incorporation at each position designated as deuterium in Compound (I-1) is at least 90%, at least 95%, or at least 97%.

In certain embodiments, the compound of Formula I is Compound (I-2):

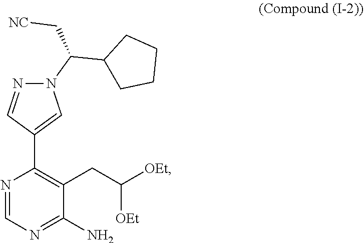 [SMILES: CCOC(Cc1c(N)ncnc1-c1cnn([C@H](CC#N)C2CCCC2)c1)OCC]

or a pharmaceutically acceptable salt thereof. As described herein, another embodiment of the invention is a composition comprising a Compound (I-2), or a pharmaceutically acceptable salt thereof an optionally a pharmaceutically acceptable carrier. In certain embodiments, the deuterium incorporation at each position designated as deuterium in Compound (I-2) is at least 90%, at least 95%, or at least 97%.

In certain embodiments, the compound of Formula (II) is Compound (II-1d):

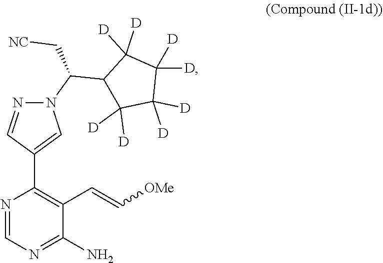

or a pharmaceutically acceptable salt thereof. As described herein, another embodiment of the invention is a composition comprising a Compound (II-1d), or a pharmaceutically acceptable salt thereof an optionally a pharmaceutically acceptable carrier. In certain embodiments, the deuterium incorporation at each position designated as deuterium in Compound (II-1d) is at least 90%, at least 95%, or at least 97%.

In certain embodiments, the compound of Formula (II) is Compound (II-2d):

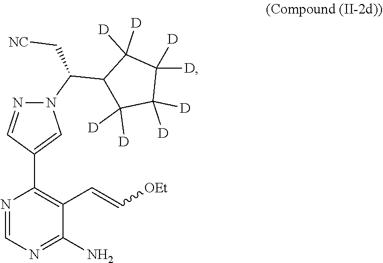

or a pharmaceutically acceptable salt thereof. As described herein, another embodiment of the invention is a composition comprising a Compound (II-2d), or a pharmaceutically acceptable salt thereof an optionally a pharmaceutically acceptable carrier. In certain embodiments, the deuterium incorporation at each position designated as deuterium in Compound (II-2d) is at least 90%, at least 95%, or at least 97%.

In certain embodiments, the compound of Formula I is Compound (II-1):

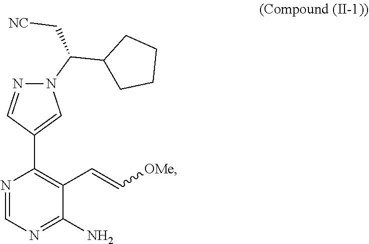 [SMILES: COC=Cc1c(N)ncnc1-c1cnn([C@H](CC#N)C2CCCC2)c1]

or a pharmaceutically acceptable salt thereof, or a composition comprising Compound (II-1), or a pharmaceutically acceptable salt thereof. As described herein, another embodiment of the invention is a composition comprising a Compound (II-1), or a pharmaceutically acceptable salt thereof an optionally a pharmaceutically acceptable carrier. In certain embodiments, the deuterium incorporation at each position designated as deuterium in Compound (II-1) is at least 90%, at least 95%, or at least 97%.

In certain embodiments, the compound of Formula (II) is Compound (II-2):

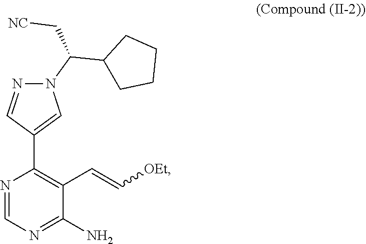 [SMILES: CCOC=Cc1c(N)ncnc1-c1cnn([C@H](CC#N)C2CCCC2)c1]

or a pharmaceutically acceptable salt thereof. As described herein, another embodiment of the invention is a composition comprising a Compound (II-2), or a pharmaceutically acceptable salt thereof an optionally a pharmaceutically acceptable carrier. In certain embodiments, the deuterium incorporation at each position designated as deuterium in Compound (II-2) is at least 90%, at least 95%, or at least 97%.

In certain embodiments, the “JAK-inhibition-responsive condition” includes, but is not limited to, diseases involving the immune system including, for example, organ transplant rejection (e.g., allograft rejection and graft versus host disease); hair loss disorders such as alopecia (including alopecia areata (AA), alopecia totalis, alopecia universalis); autoimmune diseases such as multiple sclerosis, rheumatoid arthritis, juvenile arthritis, type I diabetes, lupus, psoriasis, inflammatory bowel disease, ulcerative colitis, Crohn's disease, myasthenia gravis, immunoglobulin nephropathies, autoimmune thyroid disorders; allergic conditions such as asthma, food allergies, atopic dermatitis and rhinitis; viral diseases such as Epstein Barr virus (EBV), hepatitis B, hepatitis C, HIV, HTLV 1, varicella-zoster virus (VZV) and human papilloma virus (HPV); skin disorders such as vitiligo, psoriasis (for example, psoriasis vulgaris), atopic dermatitis, hidradenitis suppurativa, skin rash, skin irritation, skin sensitization (e.g., contact dermatitis or allergic contact dermatitis; cancer, including those characterized by solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, melanoma), hematological cancers (e.g., lymphoma, leukemia such as acute lymphoblastic leukemia, or multiple myeloma), and skin cancer such as cutaneous T-cell lymphoma (CTCL) and cutaneous B-cell lymphoma (examples of which include Sezary syndrome and mycosis fungoides; myeloproliferative disorders (MPDs) such as polycythemia vera (PV), essential thrombocythemia (ET), myeloid metaplasia with myelofibrosis (MMM), chronic myelomonocytic leukemia (CMML), hypereosinophilic syndrome (HES), systemic mast cell disease (SMCD); inflammation and inflammatory diseases, such as inflammatory diseases of the eye (e.g., iritis, uveitis, scleritis, conjunctivitis, or related disease), inflammatory diseases of the respiratory tract (e.g., the upper respiratory tract including the nose and sinuses such as rhinitis or sinusitis or the lower respiratory tract including bronchitis, chronic obstructive pulmonary disease, and the like), inflammatory myopathy such as myocarditis; systemic inflammatory response syndrome (SIRS) and septic shock; ischemia reperfusion injuries or a disease or condition related to an inflammatory ischemic event such as stroke or cardiac arrest; anorexia; cachexia; fatigue such as that resulting from or associated with cancer; restenosis; sclerodermitis; fibrosis; conditions associated with hypoxia or astrogliosis such as, for example diabetic retinopathy, cancer or neurodegeneration; gout; increased prostate size due to, e.g., benign prostatic hypertrophy or benign prostatic hyperplasia and other hair loss disorders, such as androgenetic alopecia and telogen effluvium.

In certain embodiments, the condition is selected from a hair loss disorder, polycythemia vera (PV), myelofibrosis (MF), or an acute Graft-versus-Host Disease (aGVHD).

Hair loss disorders include, without limitation, androgenetic alopecia, alopecia areata, telogen effluvium, alopecia totalis, and alopecia universalis.

Alopecia areata is an autoimmune disease that results in partial or complete loss of hair on the scalp and body that may affect up to 650,000 Americans at any given time. The scalp is the most commonly affected area, but any hair-bearing site can be affected alone or together with the scalp. Onset of the disease can occur throughout life and affects both women and men. Alopecia areata can be associated with serious psychological consequences, including anxiety and depression. There are currently no drugs approved by the U.S. Food and Drug Administration (FDA) for the treatment of alopecia areata.

In a specific embodiment, the condition is alopecia areata in a subject such as a mammalian (e.g., human) patient in need thereof. In certain embodiments, the alopecia areata is moderate to severe alopecia areata (for example, hair loss over at least 30% of the scalp, hair loss over at least 40% of the scalp, or hair loss over at least 50% of the scalp).

In certain embodiments, the subject is a human. In one embodiment, the subject is a human 6 years of age or older.

In certain embodiments, the invention provides a method for treating a disease or disorder (e.g., a hair loss disorder such as alopecia areata) that can be treated by compounds that modulate (e.g., inhibit) the activity of a JAK (JAK1 and/or JAK2), comprising administering to a mammalian subject an effective amount of any compound described herein (e.g., a compound of Formula (I) or Formula (II)) or a pharmaceutically acceptable salt thereof, once or twice per day, wherein the amount of the compound being administered, or a pharmaceutically acceptable salt thereof, is sufficient to result in the administration to the subject (e.g., a human subject) an amount of the active metabolite represented by Formula (III), for example ruxolitinib or CTP-543, that is in the range of about 4 mg/day to about 50 mg/day, for example, about 5 mg/day, about 10 mg/day, about 20 mg/day, about 30 mg/day, about 40 mg/day, or about 50 mg/day. In certain embodiments, the active metabolite is ruxolitinib. In certain embodiments, the active metabolite is CTP-543. In certain embodiments, e.g., when the active metabolite is CTP-543, the amount of the administered compound or a pharmaceutically acceptable salt thereof, is sufficient to result in the amount of the active metabolite that is about 4 mg/day, 8 mg/day, 16 mg/day, 32 mg/day or 48 mg/day. In certain embodiments, e.g., when the active metabolite is CTP-543, the amount of the administered compound or a pharmaceutically acceptable salt thereof is sufficient to result in the amount of the active metabolite that is 8 mg/day, 16 mg/day, 24 mg/day, or 32 mg/day. In certain embodiments, e.g., when the active metabolite is CTP-543, the amount of the administered compound, or a pharmaceutically acceptable salt thereof, is sufficient to result in the amount of the active metabolite that is equivalent to 10.6 mg/day of the phosphate salt of the active metabolite, e.g., administered as a twice daily dose. In certain embodiments, e.g., when the active metabolite is CTP-543, the amount of the administered compound or a pharmaceutically acceptable salt thereof, is sufficient to result in the amount of the active metabolite that is equivalent to 21.1 mg/day of the phosphate salt of the active metabolite, e.g., administered as a twice daily dose.

In certain embodiments, e.g., when the active metabolite is CTP-543, the amount of the administered compound or a pharmaceutically acceptable salt thereof, is sufficient to result in the amount of the active metabolite that is equivalent to 31.6 mg/day of the phosphate salt of the active metabolite, e.g., administered as a twice daily dose. In certain embodiments, e.g., when the active metabolite is CTP-543, the amount of the administered compound or a pharmaceutically acceptable salt thereof, is sufficient to result in the amount of the active metabolite that is equivalent to 42.2 mg/day of the phosphate salt of the active metabolite, e.g., administered as a twice daily dose.

In certain embodiments, the subject is a human. In one embodiment, the subject is a human 6 years of age or older. Preferably, the administered compound or a pharmaceutically acceptable salt thereof (such as the phosphate salt), is administered orally at any of the foregoing dosages. Preferably, the administered compound or a pharmaceutically acceptable salt thereof, is administered orally at any of the foregoing dosages in a pharmaceutical formulation which can be a tablet.

In one embodiment, the compound is administered orally once a day. In other embodiments, the compound is administered orally twice per day.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician.

The administration of compounds described herein or a pharmaceutically acceptable salt thereof (such as the phosphate salt), can continue for as long as necessary a disorder, such as a hair loss disorder, e.g., for one week, two weeks, one month, two months, three months, four months, six months, one year, two years, five years, ten years, or longer.

The efficacy of treatment of hair loss disorders such as alopecia areata can be measured in a variety of ways, some of which are known in the art. For example, the “severity of alopecia tool”, otherwise known as SALT, is a validated assessment scale—developed by the National Alopecia Areata Foundation working committee—to evaluate the degree of hair loss. See, e.g., Olsen E A, Hordinsky M K, Price V H, et al. Alopecia areata investigational assessment guidelines—Part II. J Am Acad Dermatol 2004: 51: 440-447 (incorporated herein by reference). The SALT score is calculated for a patient by measuring the percentage of hair loss in each of the 4 areas of the scalp and adding the total to achieve a composite score. Hair regrowth is reflected by a decrease in the SALT score. For example, no hair on the scalp would have a SALT score of 100 while complete hair regrowth would be a SALT score of 0. In certain embodiments, methods of treatment as described herein can provide a SALT score improvement of at least 10 points after treatment (for example, from a SALT score of 100 prior to treatment to a SALT score of 90 after treatment). In further embodiments, methods of treatment as described herein can provide a SALT score improvement of at least 20 points, 30 points, 40 points, 50 points, 60 points, 70 points, 80 points, 90 points, or 100 points. In certain embodiments, methods of treatment as described herein can provide after treatment at least a 20% improvement from baseline in the patient's SALT score, or at least a 30% improvement from baseline in the patient's SALT score, or at least a 40% improvement from baseline in the patient's SALT score, or at least a 50% improvement from baseline in the patient's SALT score, or at least a 60% improvement from baseline in the patient's SALT score, or at least a 70% improvement from baseline in the patient's SALT score.

In certain embodiments, treatment is continued for a period of at least four weeks, or at least 8 weeks, or at least 12 weeks, or at least 16 weeks, or at least 20 weeks, or at least 24 weeks, or at least 28 weeks, or at least 32 weeks, or at least 36 weeks, or at least 40 weeks, or at least 44 weeks, or at least 48 weeks, or at least 52 weeks.

### Combination Therapy

In certain embodiments, compounds described herein or a pharmaceutically acceptable salt thereof, can be administered in combination with a second therapeutic agent. Preferably, the second therapeutic agent is an agent useful in the treatment of JAK inhibition responsive disorders such as hair loss disorders or autoimmune conditions, such as inhibitors of JAK1, JAK2, or JAK3, and/or STAT1. Such inhibitors include ruxolitinib, tofacitinib, baricitinib, filgotinib, and the like. Other orally administered second therapeutic agents include agents used in the treatment of alopecia areata, including, for example, oral corticosteroids.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); the FDA-approved labeling information for ruxolitinib and tofacitinib; and clinical trial information for baricitinib and filgotinib, each of which references are incorporated herein by reference in their entirety.

It is expected that some of the second therapeutic agents referenced above will act synergistically with the compounds of this invention. When this occurs, it will allow the effective dosage of the second therapeutic agent and/or of any of the compounds described herein, or a pharmaceutically acceptable salt thereof, to be reduced from that required in a monotherapy. This has the advantage of minimizing toxic side effects of either the second therapeutic agent or a compound described herein, or a pharmaceutically acceptable salt thereof, synergistic improvements in efficacy, improved ease of administration or use and/or reduced overall expense of compound preparation or formulation.

The choice of second therapeutic agent may be made from any second therapeutic agent known to be useful for treatment of hair loss disorders such as alopecia areata. The choice of second therapeutic agent is also dependent upon the particular disease or condition to be treated. Examples of second therapeutic agents that may be employed in the methods of this invention are those set forth above for use in combination compositions comprising any of the compounds described herein, or a pharmaceutically acceptable salt thereof, and a second therapeutic agent. Additional therapeutic agents include agents used in the treatment of alopecia areata, including, for example, topical minoxidil, injected corticosteroids, and anthralin cream or ointment.

The term “co-administered” as used herein means that the second therapeutic agent may be administered together with any of the compounds described herein or a pharmaceutically acceptable salt thereof, as part of a single dosage form (such as a composition of this invention comprising a compound of the invention and an second therapeutic agent as described above) or as separate, multiple dosage forms. Alternatively, the additional agent may be administered prior to, consecutively with, or following the administration of any of the compounds described herein, or a pharmaceutically acceptable salt thereof. In such combination therapy treatment, both a compound described herein, or a pharmaceutically acceptable salt thereof, and the second therapeutic agent(s) are administered by conventional methods. The administration of a composition of this invention, comprising both any of the compounds described herein, or a pharmaceutically acceptable salt thereof, and a second therapeutic agent, to a subject does not preclude the separate administration of that same therapeutic agent, any other second therapeutic agent or any of the compounds described herein, or a pharmaceutically acceptable salt thereof, to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of any of the compounds described herein (e.g., the Compounds of Formulas (I) or (II), or a pharmaceutically acceptable salt thereof), is less than its effective amount would be where the second therapeutic agent is not administered. In another embodiment, the effective amount of the second therapeutic agent is less than its effective amount would be where any of the compounds described herein, or a pharmaceutically acceptable salt thereof, is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of any of the compounds described herein, or a pharmaceutically acceptable salt thereof, alone or together with one or more of the above-described second therapeutic agents in the manufacture of a medicament, either as a single composition or as separate dosage forms, for treatment or prevention in a subject of a disease, disorder or symptom set forth above. Another aspect of the invention is any of the compounds described herein, or a pharmaceutically acceptable salt thereof, for use in the treatment or prevention in a subject of a disease, disorder or symptom thereof delineated herein.

Another aspect of the invention is a pharmaceutical composition comprising a compound of Formula (I) or Formula (II) or a pharmaceutically acceptable salt thereof, together with a pharmaceutically acceptable carrier or diluent. The amount of the compound of Formula (I) or Formula (II) present in the pharmaceutical composition is an amount sufficient provide the active metabolite in the range of about 4 mg/day to about 50 mg/day, for example, about 5 mg/day, about 10 mg/day, about 20 mg/day, about 30 mg/day, about 40 mg/day, or about 50 mg/day. In certain embodiments, the amount of the compound or a pharmaceutically acceptable salt thereof, is sufficient to result in the amount of the active metabolite that is about 4 mg/day, 8 mg/day, 16 mg/day, 32 mg/day or 48 mg/day. In certain embodiments, the amount of the compound or a pharmaceutically acceptable salt thereof is sufficient to result in the amount of the active metabolite that is 8 mg/day, 16 mg/day, 24 mg/day, or 32 mg/day. In certain embodiments, the active metabolite is ruxolitinib. In certain embodiments, the active metabolite is CTP-543.

Another aspect of the invention is a unit dose form comprising a compound of Formula (I) or Formula (II) or a pharmaceutically acceptable salt thereof together with a pharmaceutically acceptable carrier or diluent. The amount of the compound of Formula (I) or Formula (II) present in the unit dose form is an amount sufficient provide the active metabolite in the range of about 4 mg/day to about 50 mg/day, for example, about 5 mg/day, about 10 mg/day, about 20 mg/day, about 30 mg/day, about 40 mg/day, or about 50 mg/day. In certain embodiments, the amount of the compound or a pharmaceutically acceptable salt thereof, is sufficient to result in the amount of the active metabolite that is about 4 mg/day, 8 mg/day, 16 mg/day, 32 mg/day or 48 mg/day. In certain embodiments, the amount of the compound or a pharmaceutically acceptable salt thereof is sufficient to result in the amount of the active metabolite that is 8 mg/day, 16 mg/day, 24 mg/day, or 32 mg/day. In certain embodiments, the active metabolite is ruxolitinib. In certain embodiments, the active metabolite is CTP-543. In certain embodiments, the unit dose form is a tablet.

### Synthesis of Compounds Described Herein

### I. Synthesis of Compounds Represented by Formula (I)

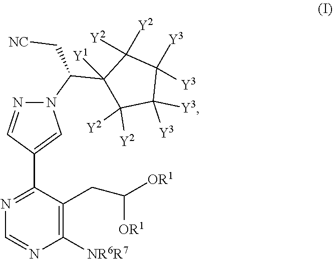

The synthesis of compounds represented by structural formula (I), or a pharmaceutically acceptable salt thereof (such as the phosphate salt) can be readily achieved by the methods described in international patent Application No. PCT/US2020/017093, published as WO2020/163653, the teachings of which are incorporated herein by reference, with appropriate modifications.

Such synthetic procedures are exemplified by the synthetic schemes resulting in compounds (I-1) and (I-1d) designated in WO2020/163653 as compounds (E-6) and (E-6′), respectively:

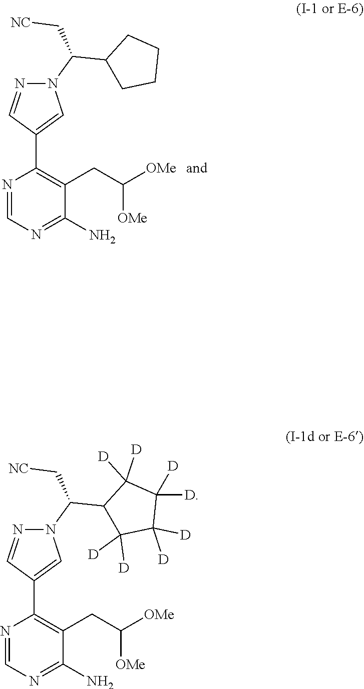

The following synthetic scheme can be employed to produce compound (I-1):

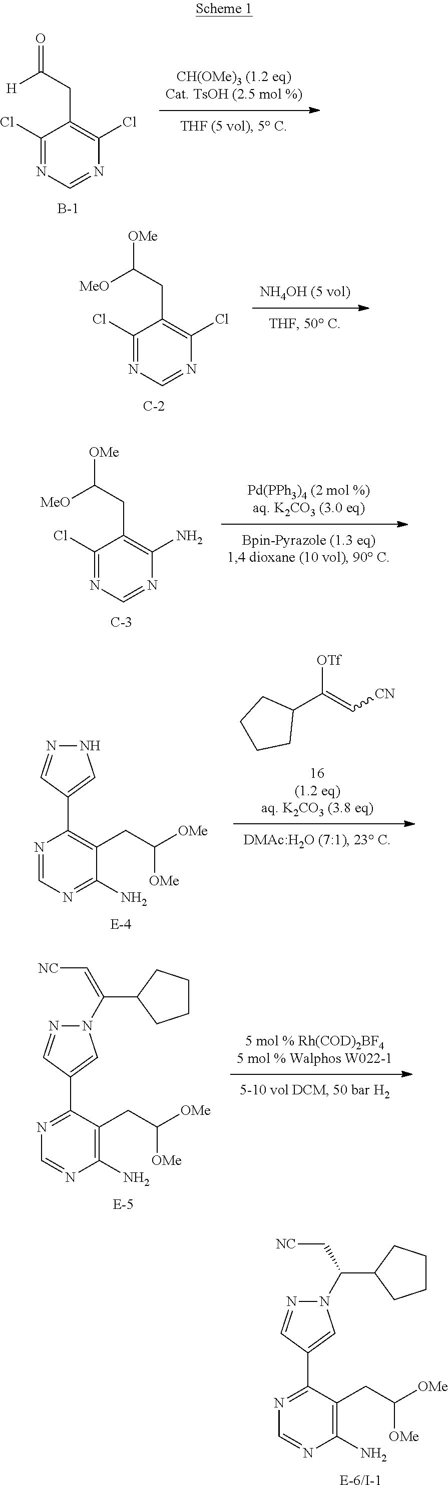

The synthesis of intermediate E-4 may be achieved alternatively by the methods described in international patent Application No. PCT/US2021/039653, published as WO2022/006136, the entire contents of which are incorporated herein by reference.

Compound 16 in Scheme 1 can be obtained according to the following synthetic scheme:

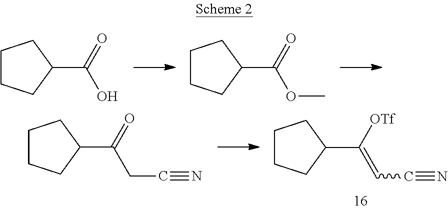

Modification of Scheme 2 (Scheme 2′) results in a deuterated analog of compound 16, which can be used to produce deuterated analogs of compound (I-1), such as compound (I-1d), according to Scheme 3.

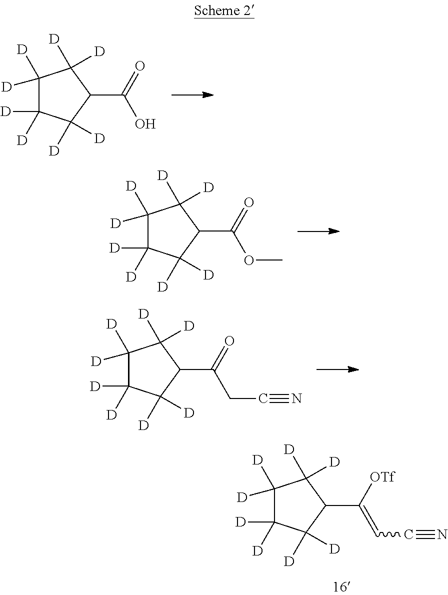

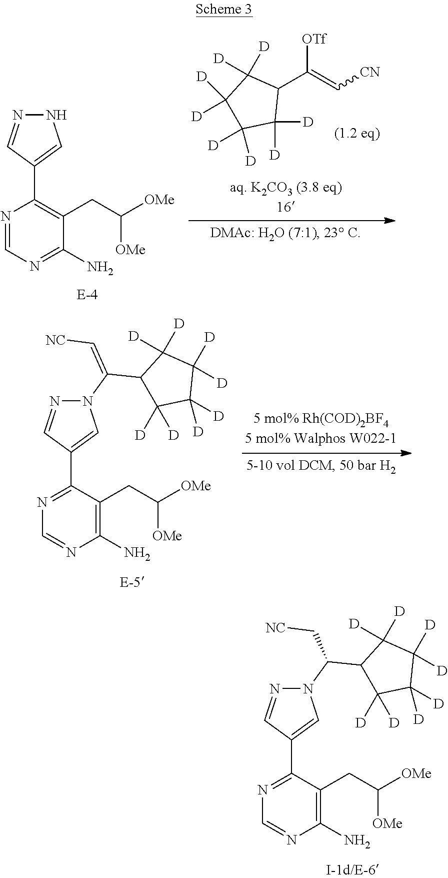 [SMILES: COC(Cc1c(N)ncnc1-c1cn[nH]c1)OC ; [2H]C1([2H])C(C(=CC#N)OS(=O)(=O)C(F)(F)F)C([2H])([2H])C([2H])([2H])C1([2H])[2H] ; [2H]C1([2H])C(/C(=C/C#N)n2cc(-c3ncnc(N)c3CC(OC)OC)cn2)C([2H])([2H])C([2H])([2H])C1([2H])[2H] ; [2H]C1([2H])C([C@@H](CC#N)n2cc(-c3ncnc(N)c3CC(OC)OC)cn2)C([2H])([2H])C([2H])([2H])C1([2H])[2H]]

A similar synthetic route, described in WO2020/163653, can produce compounds (I-2) and (I-2d):

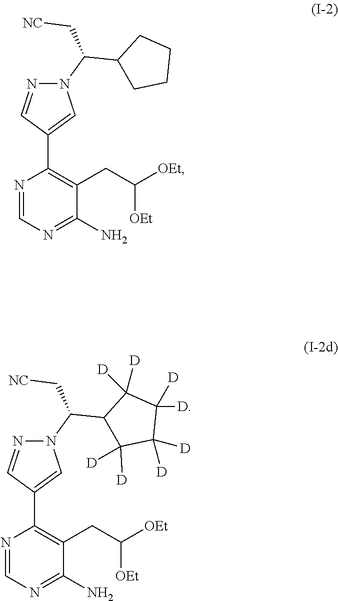

Namely, the following Scheme 4, can be used to produce compound (I-2).

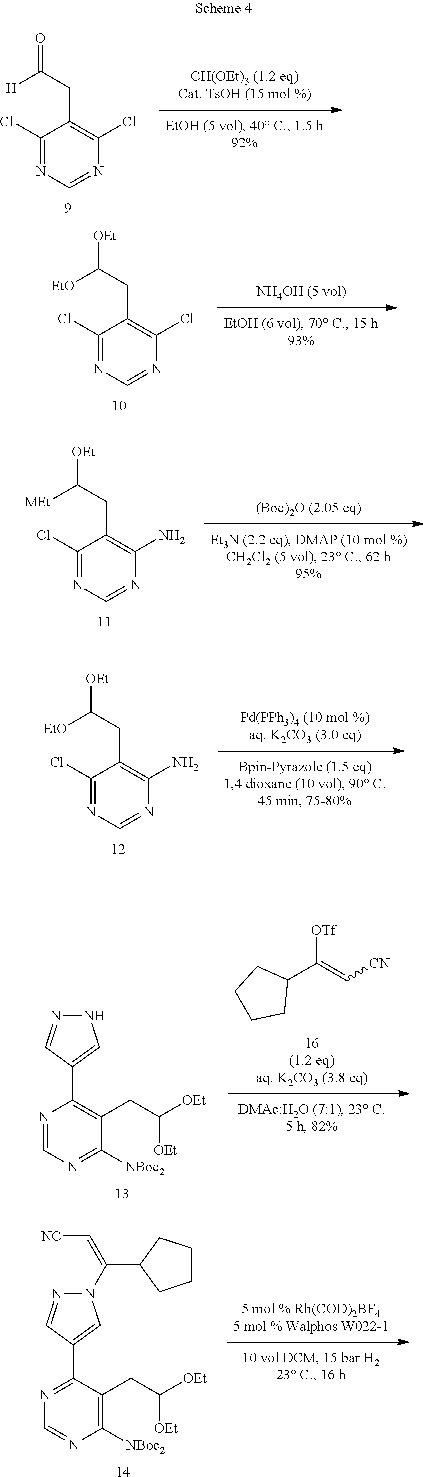

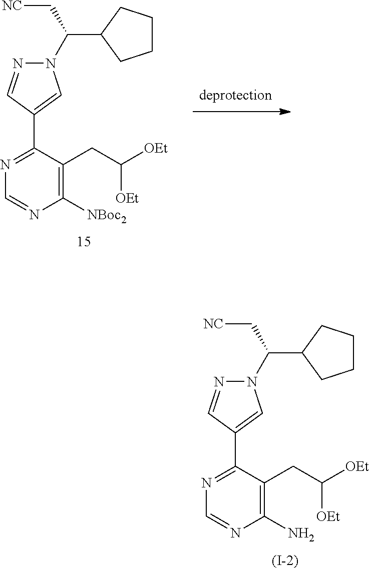

Replacement of compound 16 with compound 16′ in Scheme 4 will result in the final product being compound (I-2d).

### II. Synthesis of Compounds of Formula (II)

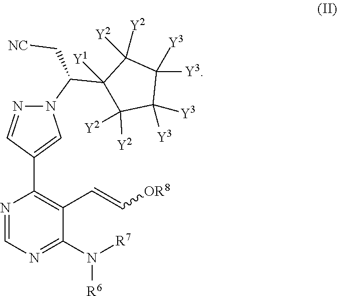

The synthesis of compounds represented by structural Formula (II), or a pharmaceutically acceptable salt thereof (such as the phosphate salt) can be readily achieved by the methods known to a person of ordinary skill in the art, as exemplified by the synthesis of Compound (II-1) shown below in Scheme 5:

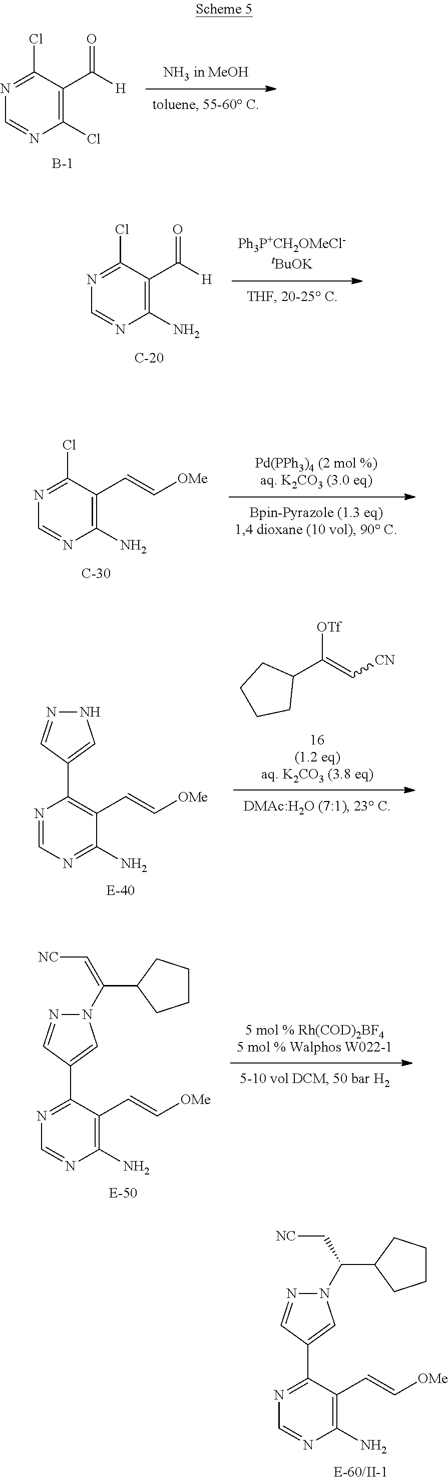

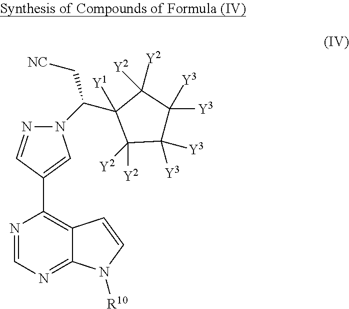

Synthesis of compounds represented by structural formula (IV) can be easily accomplished by a person skilled in the art. In particular, a synthetic route suitable for arriving at a compound of formula (IV) is described in the publication CN107915738A, the entire teachings of which is incorporated herein by reference.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

For example, Scheme 6 can be used:

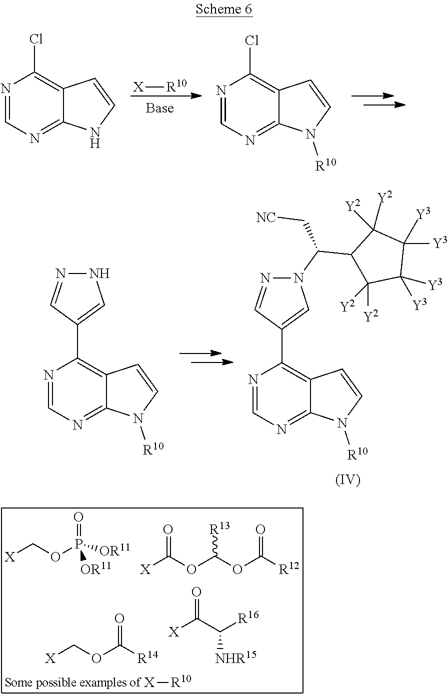

In Scheme 6:
R¹² and R¹⁴, each independently, is a C₁-C₆ alkyl, a C₆-C₁₈ aryl, a (C₆-C₁₈)aryl-(C₁-C₃)alkyl, a 5-18-member heteroaryl, a C₃-C₆ cycloalkyl, a 5-7-member heterocyclyl; an —O—(C₁-C₆)alkyl; an —N-(mono or di)(C₁-C₆)alkyl; or a —O—(C₆-C₁₈)aryl, each of which can be optionally substituted;
R¹³ and R¹⁶, each independently, is a C₁-C₆ alkyl, a C₆-C₁₈ aryl, a (C₆-C₁₈)aryl-(C₁-C₃)alkyl, a 5-18-member heteroaryl, a C₃-C₆ cycloalkyl, or a 5-7-member heterocyclyl, each of which can be optionally substituted;
R¹¹, for each occurrence independently, is a C₁-C₆ alkyl, a C₆-C₁₈ aryl, a (C₆-C₁₈)aryl-(C₁-C₃)alkyl, each of which can be optionally substituted;
R¹⁵ is a nitrogen-protecting group, such as defined above and exemplified below with respect to R¹⁰; and
R¹⁰ is any nitrogen-protecting group, and in certain embodiments is a group that can be cleaved in vivo. In addition to the protecting groups recited above, permitted values of R¹⁰ include protecting group selected from pivaloyloxymethyl (POM), 2-(trimethylsilyl)ethoxymethyl (SEM), benzyl (Bn), p-methoxybenzyl (PMB), 3,4-dimethoxybenzyl (DMPM), 2,4-dimethoxybenzyl, benzenesulfonyl, tosyl (Ts), 1-butoxycarbonyl (BOC), methoxycarbonyl (MOC), benzyloxycarbonyl (CBz), 1-naphthalenesulfonate (1-napsyl), 4-nitrobenzenesulfonyl (p-nosyl), and 2,4,6-trimethylphenylsulfonyl.

In Scheme 6, X is a suitable leaving group, such as halide (such as Cl, Br, or I), mesylate, triflate, and the like. A person of skill in the art can easily determine the type of a protecting group that can be used. See, e.g., Peter G. M. Wuts, Theodora W. Greene, “Greene's Protective Groups in Organic Synthesis”, Fourth Ed. (2006), Print ISBN:9780471697541, the relevant teachings of which are incorporated herein by reference. In Scheme 6, some examples of X—R¹⁰ can include protecting groups (e.g., at positions R¹¹ and R¹⁵) in order to be installed and/or to provide stability throughout the remaining steps of the synthesis. In those cases the protecting groups can be removed as a final synthetic step to afford the active prodrug (where R¹¹ and/or R¹⁵ would be H).

Alternatively, it is possible to prepare compounds of Formula IV directly from a compound of Formula (III) by reacting such a compound with X—R₁₀ (with or without protecting groups as needed), as shown in Scheme 7.

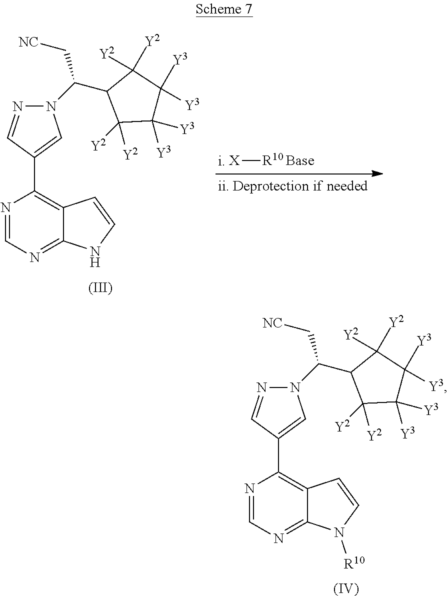

where X was defined above with respect to Scheme 6.

In yet another alternative, a compound of Formula (IV) can be accomplished according to Scheme 8:

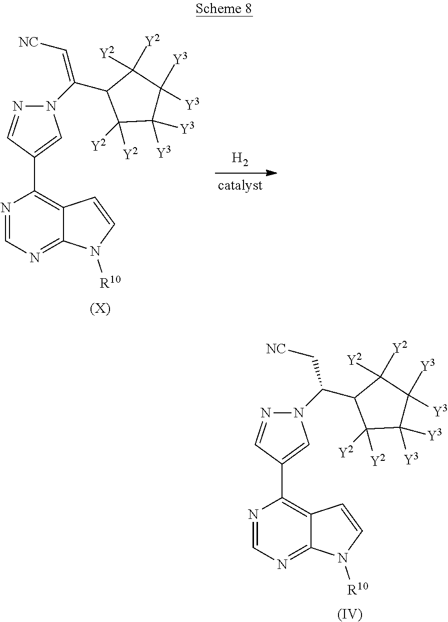

The synthesis of compounds of Formula (X) can be readily achieved by synthetic chemists of ordinary skill by reference to the synthetic schemes disclosed, for example, in U.S. Pat. No. 8,410,265, the relevant teachings of which are incorporated herein by reference.

In Formula (X), groups R¹⁰ can be a protecting group selected from pivaloyloxymethyl (POM), 2-(trimethylsilyl)ethoxymethyl (SEM), benzyl (Bn), p-methoxybenzyl (PMB), 3,4-dimethoxybenzyl (DMPM), 2,4-dimethoxybenzyl, benzenesulfonyl, tosyl (Ts), t-butoxycarbonyl (BOC), methoxycarbonyl (MOC), benzyloxycarbonyl (CBz), 1-naphthalenesulfonate (1-napsyl), 4-nitrobenzenesulfonyl (p-nosyl), and 2,4,6-trimethylphenylsulfonyl. The hydrogenation catalyst can comprise rhodium and a chiral phosphine ligand (L) according to Formula (XX):

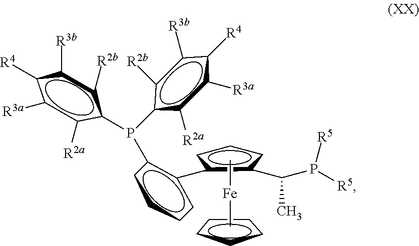

wherein each of R^{2a}, R^{2b}, R^{3a}, R^{3b}, and R⁴ is independently selected from hydrogen, methyl, methoxy, and trifluoromethyl; and R⁵ is secondary alkyl, tertiary alkyl, or cycloalkyl.

Compound of Formula (X) can be prepared from compound 40 using compound 80, according to Scheme 9:

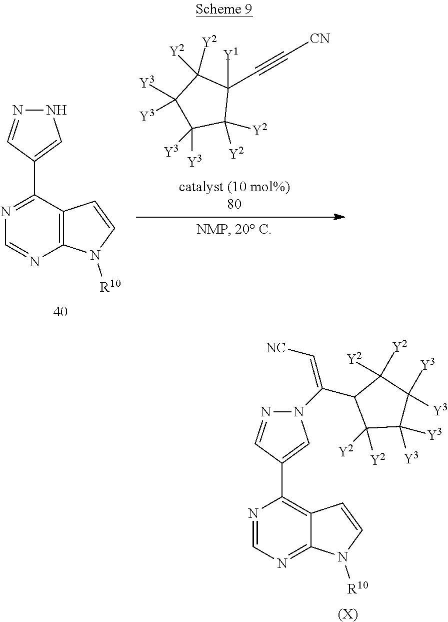

Compound 40 may be prepared in a manner analogous to those described in U.S. Pat. Nos. 9,249,149 and 8,410,265.

### Pharmaceutical Compositions

The invention also provides pharmaceutical compositions comprising an effective amount of any of the compound described herein, or a pharmaceutically acceptable salt thereof, and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament. In certain embodiments, the pharmaceutical composition is provided as a unit dose form.

The invention provides a pharmaceutical composition comprising a pharmaceutically acceptable carrier or diluent and any compound described herein or a pharmaceutically acceptable salt thereof.

In another aspect, the invention provides a pharmaceutical composition of a compound of any of Formulae (I), (II), or (IV).

In one embodiment, the invention provides a pharmaceutical composition of a compound of Formula (I), or a pharmaceutically acceptable salt thereof, the pharmaceutical composition comprising a compound of Formula (I), or a pharmaceutically acceptable salt thereof, and a pharmaceutically acceptable carrier.

In one embodiment, the invention provides a pharmaceutical composition of a compound of Formula (II), or a pharmaceutically acceptable salt thereof, the pharmaceutical composition comprising a compound of Formula (II), or a pharmaceutically acceptable salt thereof, and a pharmaceutically acceptable carrier.

In one embodiment, the invention provides a pharmaceutical composition of a compound of Formula (IV), or a pharmaceutically acceptable salt thereof, the pharmaceutical composition comprising a compound of Formula (IV), or a pharmaceutically acceptable salt thereof, and a pharmaceutically acceptable carrier.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral administration. Other formulations may conveniently be presented in unit dosage form, e.g., tablets, sustained release capsules, granules, and in liposomes, and may be prepared by any methods well known in the art of pharmacy. See, for example, Remington: The Science and Practice of Pharmacy, Lippincott Williams & Wilkins, Baltimore, MD (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In a specific embodiment, the compound is administered orally as a tablet.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added. In another embodiment, the composition is in the form of a tablet. In certain embodiments, exemplary formulations for the tablet are disclosed in U.S. Pat. No. 8,754,224, the teachings of which are herein incorporated by reference.

In a particular embodiment, a tablet formulation contains an effective amount of any compound described herein (e.g., a Compound of Formula (I) or Formula (II)) or a pharmaceutically acceptable salt thereof wherein the effective amount is sufficient to result in about 4 mg to about 50 mg of active metabolite or an equivalent amount of a pharmaceutically acceptable salt thereof (such as the phosphate salt), and the following inactive ingredients: colloidal silicon dioxide, magnesium stearate, microcrystalline cellulose, and povidone. Wet granulation followed by compression provides tablets comprising a compound of Formula (I) or Formula (II) or a pharmaceutically acceptable salt thereof.

In another embodiment, a composition of this invention comprising a compound of Formula (I) or Formula (II) or a salt thereof and a pharmaceutically acceptable carrier further comprises a second therapeutic agent. The second therapeutic agent may be selected from any compound or therapeutic agent known to have or that demonstrates advantageous properties when administered with a compound having the same mechanism of action as ruxolitinib.

Preferably, the second therapeutic agent is an agent useful in the treatment of hair loss disorders or autoimmune conditions, including inhibitors of JAK1, JAK2, or JAK3, and/or STAT1. Such inhibitors include ruxolitinib, tofacitinib, baricitinib, filgotinib, and the like. Additional thereaputic agents include agents used in the treatment of hair loss disorders such as alopecia areata, including, for example, topical minoxidil, injected corticosteroids, oral corticosteroids and anthralin cream or ointment.

In another embodiment, the invention provides separate dosage forms of the compound of Formula (I) or Formula (II), or a pharmaceutically acceptable salt thereof, and one or more of any of the above-described second therapeutic agents, wherein the compound of Formula (I) or Formula (II) or a pharmaceutically acceptable salt thereof, and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of Formula (I) or Formula (II), or a pharmaceutically acceptable salt thereof, is present in an effective amount. As used herein, an “effective amount” of the prodrug defined by structural formulas (I) and (II) is an amount sufficient to produce a therapeutically effective amount of its corresponding active metabolite upon oral administration.

The interrelationship of dosages for animals and humans (based on milligrams per meter squared of body surface) is described in Freireich et al., Cancer Chemother. Rep, 1966, 50: 219. Body surface area may be approximately determined from height and weight of the subject. See, e.g., Scientific Tables, Geigy Pharmaceuticals, Ardsley, N.Y., 1970, 537.

### EXAMPLES

### Example 1: In Vitro Study of the Conversion of a Compound of Formula (I) to its Active Metabolite, a Compound of Formula (III)

An in vitro study was conducted to test the conversion of the dibenzoyl-D-tartaric acid (DBTA) salt of Compound (I-1d) (a compound of Formula (I)) to CTP-543 (a compound of Formula (III)) when subjected to biologically-relevant conditions similar to human stomach conditions (0.1N hydrochloric acid, 37° C.).

Study Conditions: At 37° C., 28.5 mg of Compound (I-1d)·DBTA salt (equivalent to 14.6 mg of Compound (I-1d)) was added to 250 mL of 0.1N HCl while stirring. Samples were removed via syringe at 30, 60, 120 and 240 minutes, (times relevant to potential human gastric residence time), then were filtered through a syringe filter and analyzed by HPLC. Results of the HPLC analyses are shown in the table below.

Compound (I-1d) Conversion to CTP-543 in 0.1N HCl at 37° C.

| | CTP-543 Peak | CTP-543 Concentration | % Conversion |
|---|---|---|---|
| Time (min) | Area | (mg/mL) | to CTP-543 |
| 30 | 43.92779 | 0.004 | 8 |
| 60 | 54.81429 | 0.005 | 10 |
| 120 | 85.10751 | 0.008 | 16 |
| 240 | 113.98502 | 0.011 | 22 |

Result: Conversion of Compound (I-1d) to CTP-543 was observed using biologically-relevant test conditions.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

What is claimed is:

1. A method of treating a JAK-inhibition-responsive condition in a subject in need thereof, the method comprising:
administering to the subject an effective amount of a compound represented by structural Formula (I) or (II): 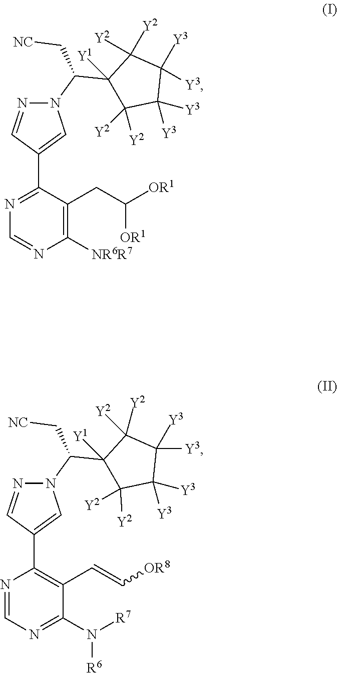
or a pharmaceutically acceptable salt thereof, wherein for each occurrence independently:
Y¹ is hydrogen or deuterium;
Y² is the same and is hydrogen or deuterium;
Y³ is the same and is hydrogen or deuterium;
R⁸ is a C₁-C₆ alkyl;
each R¹ independently is a C₁-C₆ alkyl, or the two R¹s, taken together with the oxygen atoms to which they are attached, form a 5- or 6-membered heterocyclic ring; and R⁶ and R⁷, for each occurrence are independently selected from H and a nitrogen protecting group.

2. The method of claim 1, wherein Y¹ is hydrogen and each of Y² and Y³ is deuterium.

3. The method of claim 1, wherein each of Y¹, Y², and Y³ is hydrogen.

4. The method of any one of claims 1-3, wherein each R¹ and R⁸, independently, is methyl or ethyl.

5. The method of any one of claims 1-4, wherein each R¹ is methyl.

6. The method of any one of claims 1-3, wherein each R¹ is ethyl.

7. The method of any one of claims 1-3, wherein R⁸ is methyl.

8. The method of any one of claims 1-3, wherein R⁸ is ethyl.

9. The method of any of the preceding claims, wherein R⁶, for each occurrence independently, is H.

10. The method of any of the preceding claims, wherein, for each occurrence R⁶ is H and R⁷ is a nitrogen protecting group.

11. The method of any one of the preceding claims, wherein the nitrogen protecting group is selected from t-butoxycarbonyl (Boc), triflyl (Tf, SO₂—CF₃), trifluoroacetyl (F₃—Ac), and trityl (Tr, CPh₃).

12. The method of claim 11, wherein the protecting group is a t-butoxycarbonyl group.

13. The method of any one of claims 1-3, wherein the compound of Formula (I) is Compound (I-1d): 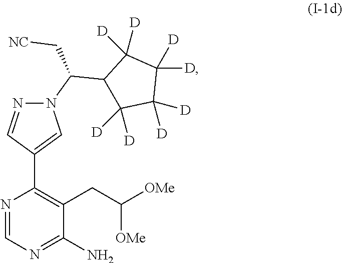
or a pharmaceutically acceptable salt thereof.

14. The method of any one of claims 1-3, wherein the compound of Formula (I) is Compound (I-2d): 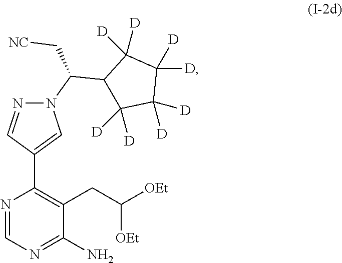
or a pharmaceutically acceptable salt thereof.

15. The method of any one of claims 1-3, wherein the compound of Formula (I) is Compound (I-1): 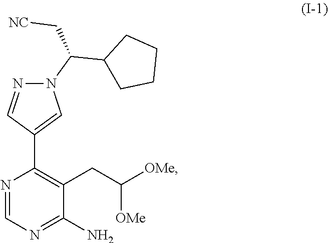 [SMILES: COC(Cc1c(N)ncnc1-c1cnn([C@H](CC#N)C2CCCC2)c1)OC]
or a pharmaceutically acceptable salt thereof.

16. The method of any one of claims 1-3, wherein the compound of Formula (I) is Compound (I-2): 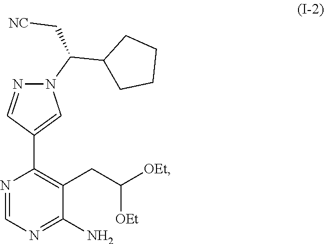 [SMILES: CCOC(Cc1c(N)ncnc1-c1cnn([C@H](CC#N)C2CCCC2)c1)OCC]
or a pharmaceutically acceptable salt thereof.

17. The method of any one of claims 1-3, wherein the compound is of Formula (II) is Compound (II-1d): 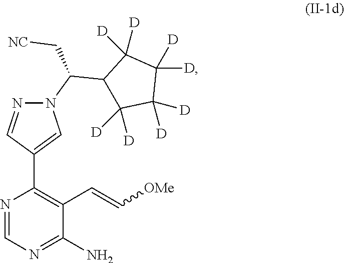
or a pharmaceutically acceptable salt thereof.

18. The method of any one of claims 1-3, wherein the compound of Formula (II) is Compound (II-2d): 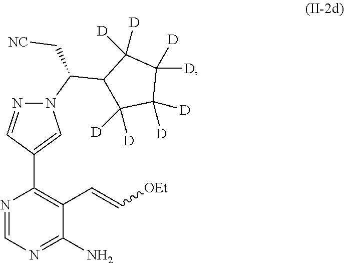
or a pharmaceutically acceptable salt thereof.

19. The method of any one of claims 1-3, wherein the compound of Formula (II) is Compound (II-1): 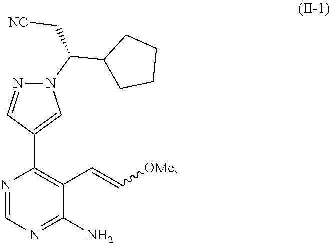 [SMILES: COC=Cc1c(N)ncnc1-c1cnn([C@H](CC#N)C2CCCC2)c1]
or a pharmaceutically acceptable salt thereof.

20. The method of any one of claims 1-3, wherein the compound of Formula (II) is Compound (II-2): 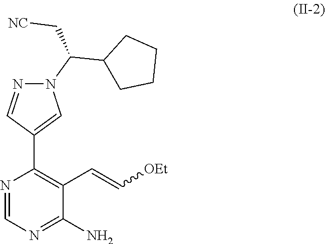 [SMILES: CCOC=Cc1c(N)ncnc1-c1cnn([C@H](CC#N)C2CCCC2)c1]
or a pharmaceutically acceptable salt thereof.

21. The method of any one of claims 1-20, wherein the JAK-inhibition-responsive condition is selected from a hair loss disorder, organ transplant rejection, multiple sclerosis, rheumatoid arthritis, juvenile arthritis, type I diabetes, lupus, psoriasis, inflammatory bowel disease, ulcerative colitis, Crohn's disease, myasthenia gravis, immunoglobulin nephropathies, autoimmune thyroid disorders, asthma, food allergies, atopic dermatitis, rhinitis, Epstein Barr virus (EBV), hepatitis B, hepatitis C, HIV, HTLV 1, varicella-zoster virus (VZV), human papilloma virus (HPV), skin rash, skin irritation, skin sensitization, vitiligo, hidradenitis suppurativa, prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, melanoma, lymphoma, leukemia, cutaneous T-cell lymphoma (CTCL) and cutaneous B-cell lymphoma, polycythemia vera (PV), essential thrombocythemia (ET), myeloid metaplasia with myelofibrosis (MMM), chronic myelomonocytic leukemia (CMML), hypereosinophilic syndrome (HES), systemic mast cell disease (SMCD), iritis, uveitis, scleritis, conjunctivitis, rhinitis, sinusitis, bronchitis, chronic obstructive pulmonary disease, myocarditis, systemic inflammatory response syndrome (SIRS), septic shock, ischemia reperfusion injuries, stroke, cardiac arrest, anorexia, cachexia, fatigue, restenosis, sclerodermitis, fibrosis, diabetic retinopathy, neurodegeneration, gout, benign prostatic hypertrophy and benign prostatic hyperplasia.

22. The method of claim 21, wherein the condition is a hair loss disorder, polycythemia vera (PV), myelofibrosis (MF), or an acute Graft-versus-Host Disease (aGVHD).

23. The method of claim 22, wherein the hair loss disorder is alopecia areata.

24. The method of any one of claims 1-23, wherein the compound is administered orally.

25. The method of any one of claims 1-24, wherein the compound is administered in a pharmaceutical formulation which is a tablet.

26. The method of any one of claims 1-25, wherein the compound is administered once a day.

27. The method of any one of claims 1-25, wherein the compound is administered twice a day.

28. A method of treating a JAK-inhibition-responsive condition in a subject in need thereof, the method comprising:
administering to the subject an effective amount of a pharmaceutical composition comprising a pharmaceutically acceptable carrier or diluent and a compound represented by structural Formula (I) or (II): 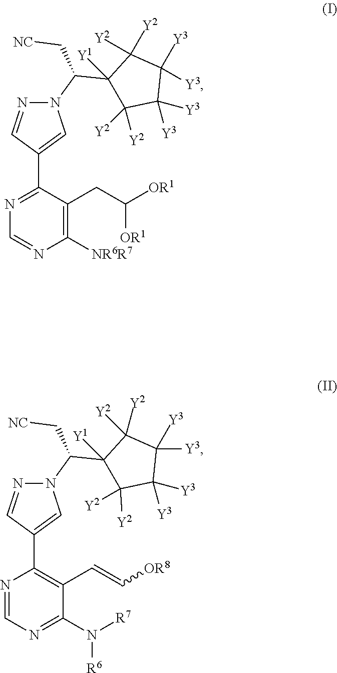
or a pharmaceutically acceptable salt thereof, wherein, for each occurrence independently:
Y¹ is hydrogen or deuterium;
Y² is the same and is hydrogen or deuterium;
Y³ is the same and is hydrogen or deuterium;
R⁸ is a C₁-C₆ alkyl;
each R¹ independently is a C₁-C₆ alkyl, or the two R¹s, taken together with the oxygen atoms to which they are attached, form a 5- or 6-membered heterocyclic ring; and R⁶ and R⁷, for each occurrence are independently selected from H and a nitrogen protecting group.

29. A method of treating a JAK-inhibition-responsive condition in a subject in need thereof, the method comprising:
administering to the subject an effective amount of a compound represented by structural Formula (IV): 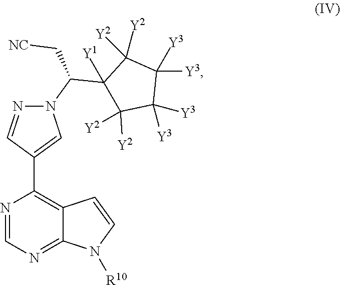
or a pharmaceutically acceptable salt thereof, wherein for each occurrence independently:
Y¹ is hydrogen or deuterium;
Y² is the same and is hydrogen or deuterium;
Y³ is the same and is hydrogen or deuterium;
R⁸ is a C₁-C₆ alkyl; and
R¹⁰ is a nitrogen-protecting group.

30. A method of treating a JAK-inhibition-responsive condition in a subject in need thereof, the method comprising:
administering to the subject an effective amount of a pharmaceutical composition comprising a pharmaceutically acceptable carrier or diluent and a compound represented by structural Formula (IV): 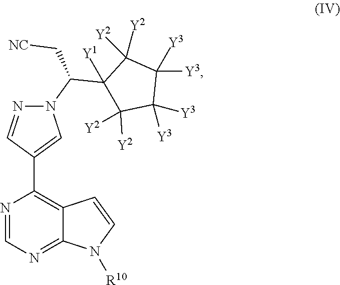
or a pharmaceutically acceptable salt thereof, wherein for each occurrence independently:
Y¹ is hydrogen or deuterium;
Y² is the same and is hydrogen or deuterium;
Y³ is the same and is hydrogen or deuterium;
R⁸ is a C₁-C₆ alkyl; and
R¹⁰ is a nitrogen-protecting group.
